(19) United States
(12) Patent Application Publication
(10) Pub. No.: US 2011/0087261 A1
(43) Pub. Date: Apr. 14, 2011

(54) Device and Method for Transseptal Puncturing

(75) Inventors: Frederik Henricus Mattheus Wittkampf, Lage Vuursche (NL); Helmert Van Wessel, Elst (NL)

(73) Assignee: UMC UTRECHT HOLDING B.V., Utrecht (NL)

(21) Appl. No.: 12/921,638

(22) PCT Filed: Sep. 4, 2008
(86) PCT No.: PCT/EP2008/007326

§ 371 Date: Jan. 3, 2011

Related U.S. Application Data
(60) Provisional application No. 61/068,766, filed on Mar. 11, 2008.

(51) Int. Cl. A61B 17/34

(52) U.S. Cl. 606/185

(57) ABSTRACT

Device for a medical procedure within the vasculature of a patient, comprising puncturing means provided at a distal end of the device for puncturing tissue in a supported state, and an elongate member having a proximal end, a distal end provided with a puncturing means and a flexible length therebetween, wherein the elongate member is arranged to be advanced through the vasculature in an unsupported state of the puncturing means without damaging the walls of the vasculature.

The present invention relates to a device for use in a medical procedure, specifically for use in transseptal puncturing in the heart. The invention also relates to a method for puncturing the septum of the heart.

Various interventional cardiovascular procedures require access to the left atrial cavity. The standard transseptal puncture set consists of a needle, dilator and sheath. The needle is used to puncture the intra-atrial septum. It is hollow to allow for measurement of intracardiac pressure. After puncturing, the dilator is pushed through the septum followed by the sheath. Thereafter, the needle and dilator are removed and the sheath is used to guide a catheter to the left atrium.

The rather stiff needle has the major disadvantage that after puncturing the septum, it may puncture the atrial wall too, especially when the septum is relatively thick or flexible and much force is needed to puncture it. After correct puncture of the needle and dilator and withdrawal of the needle, the sheath, supported by the dilator, must be pushed into the left atrium and doing so the rather sharp distal end of the dilator may puncture the left atrial wall and cause pericardial effusion and tamponade.

It is an object of the invention to provide in an efficient, and/or easy to use device for use in a. medical procedure, specifically for use in transseptal puncturing in the heart.

In order to accomplish that objective, a device for use in a medical procedure within the vasculature of a patient is provided, comprising:
puncturing means provided at a distal end of the device for puncturing tissue in a supported state, and;
an elongate member having a proximal end, a distal end provided with the puncturing means and a flexible length there between, wherein the elongate member is arranged to be advanced through the vasculature in an unsupported state of the puncturing means without damaging the walls of the vasculature.

The device according to the invention provides an elongate member in the form of a puncture wire which can be used as a guiding wire. In the unsupported state, the puncturing means are prevented from damaging the wall of the vasculature, allowing advancement of said elongate member through the vasculature. The flexible length with a relative low stiffness ensures that substantially no pressure or force can be exerted by the puncturing means on the vasculature since the flexible length will deform or deflect. The flexible length is therefore not able to transfer any substantial force to the distal end provided with the puncturing means which can harm the vasculature.

According to the invention, the flexible length is sufficiently flexible such that the force needed for the flexible length to deflect and/or buckle is smaller than the force needed for the puncturing means to puncture the tissue, that is damaging the tissue, in the unsupported state. When the puncturing means would for instance engage a wall of the vasculature, the flexible length would deflect instead of the puncturing said wall. To this end, the stiffness of the flexible length, that is the resistance of the flexible length to deflection or deformation by an applied force, specifically a compressive force, is sufficiently small. This prevents any unintended damage to the walls of the vasculature in case of the puncturing means engaging said walls. It is also possible that the flexible length does not show any substantial rigidity, disallowing the flexible length to transfer any force from the proximal end to the distal end in the unsupported state.

Preferably the elongate member is flexible for a length between 1 to 15 cm, preferably between 2 to 10 cm and more preferably for a length of approximately 5 cm proximal to the puncturing means. This is a suitable length which allows the puncturing means to deflect from any encountered walls of the vasculature, preventing any damage to said walls.

When the site for puncturing, preferably the septum of the heart, is reached by the puncturing means, the puncturing means are supported allowing the puncturing means to exert force on the tissue. By moving between the unsupported and supported state of the puncturing means, the device according to the invention can be used as guiding or puncture wire, respectively. It should be noted that with the term ‘supported state’, a state of the puncturing means is meant wherein said means are substantially confined, preferably in both axial and radial direction of the elongate member such that the flexible length is prevented from deflecting. It is also possible to support the elongate member on a location proximal to the puncturing means, further preventing buckling of said elongate member when puncturing.

In one preferred embodiment of the device according to the invention, the elongate member proximal to the flexible length comprises a stiff length. The stiff length provides rigidity to the device, facilitating advancement of cardiovascular devices over the elongate member, such as dilators and/or sheaths as will be discussed more in detail. The stiff section also facilitates steering through the vasculature. Preferably, the stiff section is stiffer than conventional guiding wires.

In another preferred embodiment of the device according to the invention, the distal end of the elongate member is bent. Preferably, the flexible length comprises said bend. Such a bend directs the puncturing means away from the walls of the vasculature and/or the walls of the atrium when being advanced. The distal length of the elongate member can for instance be provided with a bend of 45 degrees or 180, degrees forming a J-shape; with respect to the axis of the elongate member. The bend can have a radius of approximately 3 to 5 mm. The elongate member comprising the bend is for this end sufficiently flexible allowing the bend to be ‘straightened’ when being moved in the supported state for puncturing.

The diameter of the elongate member is preferably between 0.5 mm and 1.2 mm, more preferably between 0.7 mm and 0.9 mm, and even more preferably the diameter is approximately 0.81 mm. The length of the elongate member is preferably approximately 180 cm. It is advantageous if the elongate member has a core fabricated from Nitinol and more preferably the member is coated with polyurethane and a hydrophilic polymer. As an alternative, the elongate member can be hollow. It is also possible to provide the elongate member with an electric conductive core.

It is advantageously when the tubular member comprises a substantially smooth outer surface facilitating the advancement of said member and/or preventing any damage to the vasculature. In case the flexible length has a diameter different from the stiff length, the transition between the lengths preferably comprises a tapering length.

In another preferred embodiment of the device according to the invention the puncturing means comprise a stiff puncturing section distal to the flexible length comprising a sharp distal end. The stiff puncturing section facilitates the supporting of the puncturing means and provides a suitable puncturing ability. Preferably the puncturing section has a length between 1 and 10 mm, preferably between 3 and 7 mm, and more preferably of approximately 5 mm.

More preferably, the sharp distal end comprises at least one oblique face under an angle with the axis of the elongate member between 20 and 70 degrees, preferably between 35 and 55 degrees, and more preferably of approximately 45 degrees. It is advantageous when a drilling motion is used for puncturing the septum. Using a drilling motion, the force in a perpendicular direction to surface of the septum can be minimized, thereby minimizing the chance of puncturing the other atrial wall after punctuation.

It should however be noted that also alternative puncturing means than means for drilling can be used with the device according to the invention such as cutting means, for instance a knife, a drilling head structure or punctuation means such as a needle.

In another preferred embodiment of the device according to the invention the elongate member is provided with pressure sensing means at a distal location. This allows pressure measurements for instance after puncturing to ensure in proper puncturing. The pressure sensing means can be electronic, wherein a suitable conduit is provided in the elongate member, or based on hydrostatic pressure, wherein the elongate member is hollow allowing fluid communication between the distal end and the proximal end of the elongate member for pressure measurements using a conventional pressure monitor.

In another preferred embodiment of the device according to the invention the supporting means and the elongate member are arranged to move between the unsupported state wherein the puncturing means are unsupported and the supported state wherein the puncturing means are supported by the supporting means allowing puncturing of the tissue. By moving the supporting means relative to the elongate member, the puncturing means can be moved between the supported and unsupported state. The supporting means are arranged to confine the puncturing means in at least one of the axial or the radial direction, facilitating puncturing. In the unsupported state, the puncturing means are held by the flexible length allowing substantially any movement in the axial and radial direction with respect to elongate member. This prevents the puncturing means from inflicting damage to the vasculature.

Preferably, the supporting means comprise a tubular member movable over the elongate member, wherein at least a distal section of the tubular member has an inner diameter suitable for supporting the puncturing means in the supported state.

By moving a tubular member, preferably a dilator, in the proximity of the distal end of the elongate member and more specifically near the puncturing means, the latter are confined preventing deflection of the distal end. The corresponding inner diameter of the tubular member to the outer diameter of the puncturing means ensures a close fit of the puncturing means in the supporting means ensuring easy advancement. Moreover, the corresponding diameters prevent the tubular member from unintentionally moving distally over the puncturing means, exposing the tubular member. More preferably, the inner diameter of at least the distal, narrow end of the tapered dilator corresponds to the inner diameter of the stiff puncturing section. Using a dilator as supporting means provides a compact device, since no additional dilator has to be advanced for widening the puncture. More preferably, the tubular member is further arranged to support the elongate member on a proximal location with respect to the puncturing means. This further prevents the elongate member from buckling.

In another preferred embodiment, the tubular member is provided with a bend, wherein the tubular member is bent with an angle with respect to the axis of the elongate member between 25° and 65°, preferably approximately 45°. The bend in the tubular member, preferably a dilator, can be used to manoeuvre the dilator and elongate member combination on the septum as will be discussed in more detail below. Preferably, the tubular member is bent at a distance proximal to the distal end of between 3 and 12 cm, more preferably of between 5 and 7 cm. Preferably, the tubular member distal to the bend extends substantially rectilinear.

In another preferred embodiment of the device according to the invention, the device further comprises a sheath movable over the elongate member. Said sheath allows the introduction of various cardiovascular devices, such as catheters. Preferably, the sheath is moveable over the dilator. And more preferably, the inner diameter of the sheath corresponds to the outer diameter of the tubular member, allowing a close fit between the two. It is possible to provide the sheath with a bend, similar to the bend for the dilator as mentioned above. Preferably, the sheath is bent at a distance of approximately 2.5 cm proximal to the distal end of the sheath.

It can also be advantageously when the sheath is a steerable sheath, allowing the practitioner to steer the distal end of the sheath by deflecting said distal end. In case a steerable sheath is used, the need for a dilator provided with a bend as discussed above is less needed, since the sheath can then provide the needed steering. Preferably, the steerable sheath is provided with a bendable distal end of approximately 5 cm and is arranged to deflect up to 180° with respect to the longitudinal axis of the sheath.

Preferably, the tubular member in the form of a dilator is provided with indications means at the proximal end, allowing the practitioner to place the tubular member, for instance a dilator, on the septum with great accuracy. The indication means can comprise an indication mark, for instance an arrow, indicating for instance the orientation of the bend provided in the distal end of the dilator. In case a sheath provided with a bend is used, said sheath is preferably provided with indication means at its proximal end.

In another preferred embodiment of the device according to the invention, the distal end of the device is provided with localization means. This allows the practitioner to localize for instance the puncturing means, the dilator and/or the sheath in the patient. It is possible to use an electronic localization system, wherein the distal end of the elongate member is provided with a conducting section, for instance the puncturing means, wherein the conducting section is connected, for instance using a conducting core of the elongate member, to a suitable localization device outside the patient.

The invention furthermore relates to a method for puncturing tissue in the vasculature of a patient, specifically for puncturing the septum extending between the left and the right atrium of the heart, using a device as described above, comprising the steps of:
advancing the puncturing means provided on the distal end of the elongate member through the vasculature in the proximity of the tissue;
supporting the puncturing means allowing puncturing of the tissue, and;
activating the puncturing means for puncturing the tissue.

It will be appreciated that the above described method for puncturing a septum is very efficient. Where in the prior art methods a guiding wire was used to advance to the site of puncturing and was then exchanged for a needle for actual puncturing, the method according to the invention lacks such exchange. By advancing the puncturing means in the unsupported state through the vasculature into the proximity of the tissue to be punctured and subsequently supporting the puncturing means, the tissue can be punctured without exchanging wires or needles.

In one preferred embodiment of the method according to the invention, the step of supporting the puncturing means comprises moving a tubular member over and relative to the elongate member to the puncturing means such that the puncturing means extend distally from the tubular member allowing puncturing of the tissue. By moving a tubular member, preferably a dilator, over the puncturing means and more preferably over the stiff puncturing section as described above, an efficient transfer from the unsupported to the supported state is achieved. The extending part of the puncturing means provides the puncturing action. Preferably the puncturing means extend from the tubular member with a length between 0.1 to 10 mm and preferably 1 to 5 mm, and more preferably approximately 3 mm.

In another preferred embodiment of the method according to the invention the step of activating the puncturing means comprises rotating the elongate member. By rotating the elongate member an efficient puncturing action is achieved, without the need to apply an excess of force onto the septum in the distal direction. The change of hitting the wall of the left atrium after puncturing is hereby reduced. Furthermore, using a rotating movement of the puncturing means, the change of accidental movement of the puncturing means due to the applied force prior to actual puncturing, e.g. by sliding of the puncturing means on the septum, is avoided. Furthermore, although preferably at least the distal end of the puncturing means is supported in the supported state, the lack of any substantial distal pressure further prevents any accidental buckling or bending of the flexible length during puncturing or drilling.

It is advantageously that the elongate member provided with a flexible length allows advancement of said member through the vasculature without damaging said vasculature, while at the same time by rotating said member an efficient puncturing method is provided. Using a rotation movement creating a drilling action, it is not necessary for the flexible length to transfer any substantial distal force for puncturing according to the prior art puncturing means

It should however be noted that rotating an elongate member provided with puncturing means on its distal end for puncturing can also be used with other elongate members, for instance elongate members lacking said flexible section.

In another preferred embodiment of the method according to the invention, the step of advancing the puncturing means comprises advancing the elongate member through the vasculature whereby the flexible length holding the puncturing means is at least partially unsupported. In this unsupported state, the puncturing means are held by the flexible length, preventing said means from inflicting damage. The elongate member is sufficiently flexible for a length such that the puncturing means are not allowed to exert a substantial force on the wall when engaging said wall. Since the length proximal to the puncturing means is flexible, the puncturing means will deflect from the engaged wall instead of puncturing it.

In another preferred embodiment of the method according to the invention, the step of advancing the puncturing means further comprises the step of advancing the elongate member at least partially in the superior vena cava, retracting the puncturing means in the tubular member and subsequently descending to the septum for puncturing. Retracting the puncturing means provides a relative stiff composition allowing the practitioner to move the distal end of the tubular member, preferably the dilator, over the wall of the right atrium onto the septum. When moving over this wall, the tubular member will ‘fall’ onto the septum. Using imaging techniques the practitioner can ensure in proper placement for puncturing. With the puncturing means retracted, said means are prevented from damaging said wall when moving towards the septum.

Preferably, for placing the dilator on the septum for puncturing, the dilator is provided with a bend. The bend allows the practitioner to steer the dilator/elongate member combination to the septum. More preferably, the dilator is provided with indications means on its distal end, for instance a radiopaque marker, allowing the practitioner to image said distal end. For steering the tubular member to the fossa ovalis, also a steerable sheath can be used. Said sheath closely fits around the dilator, allowing precise steering.

Another advantage of the device according to the invention is that when the practitioner is not able to find the ‘jump’ of the distal end of the tubular member onto the fossa ovalis, the device according the invention can be easily repositioned in the superior vena cava without the need for exchanging the needle for the guide wire as with the conventional puncture systems. Retracting the tubular member with respect to the puncturing means moves the puncturing means into the unsupported state allowing easy repositioning for another try.

In another preferred embodiment, the method further comprises the step of at least partially advancing the flexible length of the elongate member through the puncture into the left atrium after puncturing the septum. By moving the puncturing means in the left atrium, said means are moved in the unsupported state, preventing any damage in the left atrium. Preferably the elongate member is advanced into a pulmonary vein, preferably the left upper pulmonary vein. This allows the practitioner to visualize proper punctuation of the septum using conventional imaging, since the left upper pulmonary vein lies outside the contours of the heart. Furthermore, said vein lies substantially straight ahead from the puncturing said making it easy for the practitioner to advance the elongate member is said vein without any substantial steering. Preferably, the flexible length extends substantially rectilinear in the unsupported state, facilitating the advancement is said vein.

It is also advantageously to conduct a pressure measurement when advancing into the, presumably, left atrium. A pressure measurement using the pressure sensing means provided on a distal part of the elongate member can ensure a practitioner that a puncture is made from the right atrium into the left atrium based on the measured pressures.

More preferably the elongate member is advanced in the left atrium until at least a part of the stiff length extends in the left atrium. The stiff section facilitates the advancement of cardiovascular devices over the elongate member, such as the dilator and sheath. Since the length of the flexible length is preferably approximately 5 cm and the diameter of the left atrium is approximately between 4 and 6 cm, advancing the elongate member for at least said 5 cm in the left atrium would result in the advancement of a part of the stiffer section.

In another preferred embodiment, the method comprises the step of at least partially advancing the tubular member through the puncture after puncturing, wherein the tubular member comprises a dilator. The tapered dilator widens the puncture, allowing the introduction of larger devices through the puncture. Preferably, the dilator is advanced over the stiff section of the elongate member.

In another preferred embodiment of the method according to the invention, the method further comprises the step of advancing a sheath through the puncture into the left atrium. After retraction of the dilator and the elongate member, the sheath can be used to introduce for instance a catheter into the left atrium.

It can be advantageously to retract the dilator from the puncture after advancement of said dilator into the left atrium. With the puncture still widened, a cardiovascular device can be advanced next to the elongate member still extending between the right and left atrium through said puncture as guidance. It can however also be possible to advance a cardiovascular device over said elongate member. With one device present in the left atrium, for instance a first catheter, the dilator and/or the sheath is advanced back through the hole. After retraction of the elongate member and the dilator, a second cardiovascular device can be introduced in the left atrium.

It should be understood that although a method for puncturing the interatrial septum is described above, the method according to the invention can also be used for puncturing other tissue, specifically septa. The method and device according to the invention can for instance be used to puncture the ventricular septum extending between the ventricles. It is hereby advantageously when the device according to the invention is introduced in the patient in the shoulder area.

The present invention is further illustrated by the following Figures, which show a preferred embodiment of the device and method according to the invention, and are not intended to limit the scope of the invention in any way, wherein:

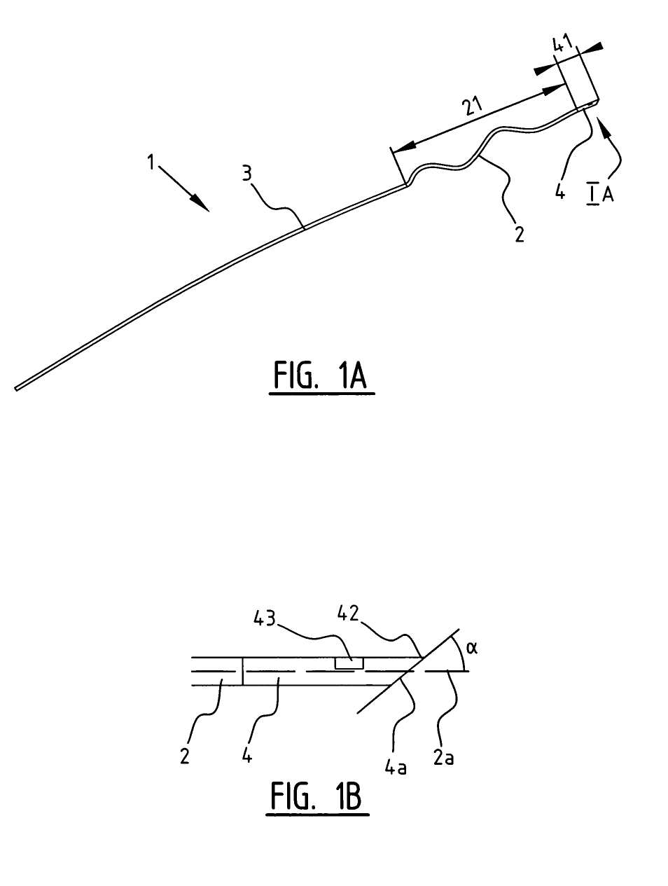

FIG. 1A schematically shows a preferred embodiment of the puncture wire according to the invention;

FIG. 1B schematically shows a detail of the distal end of the puncture wire of FIG. 1A in a direction IIa;

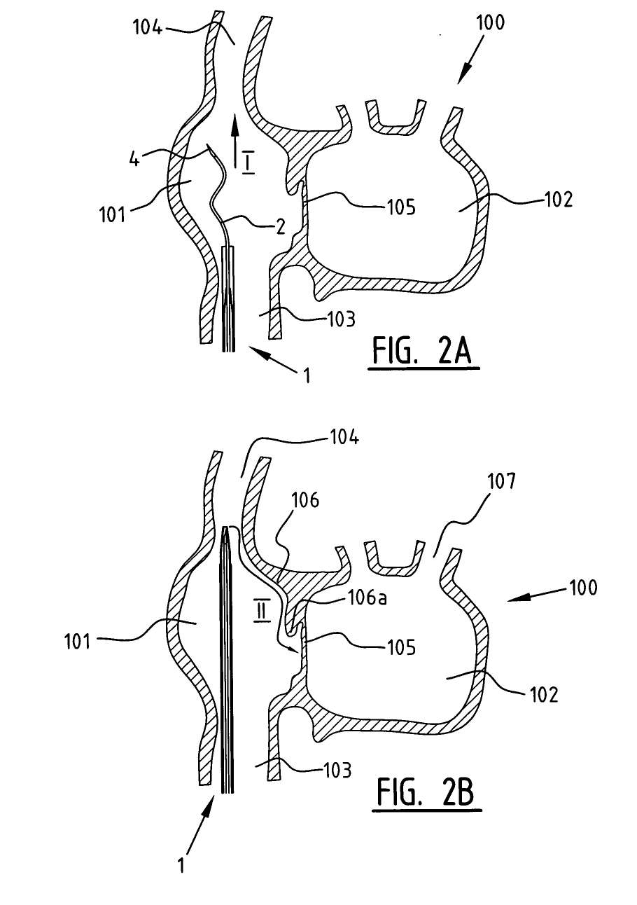

FIGS. 2A and 2B schematically show the placement of the puncture wire according to the invention to the septum, and;

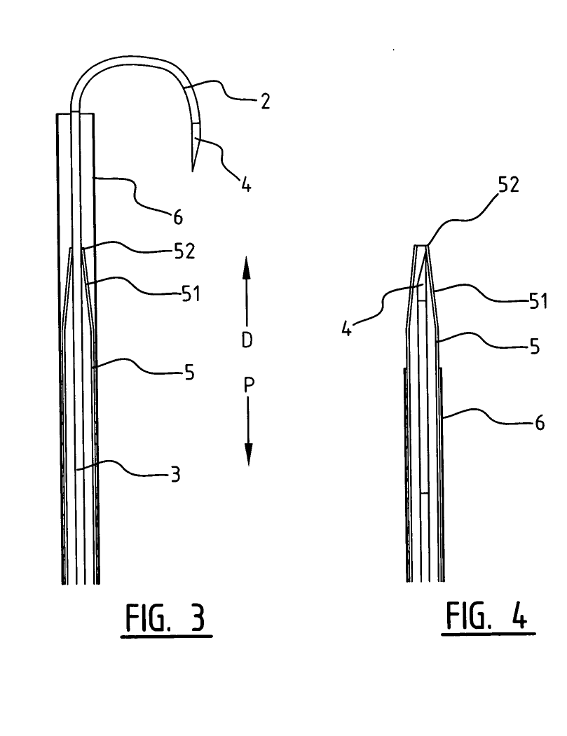

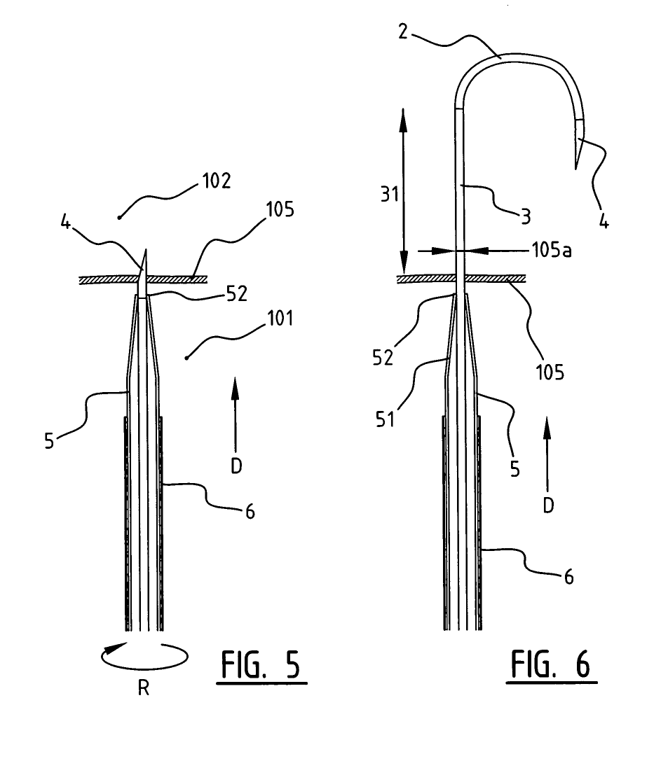

FIGS. 3-9 schematically show the different steps in the method for puncturing the septum according to the invention.

In FIG. 1A the device according to the invention in the form of a puncture wire 1 is shown. The puncture wire 1 comprises an elongate member having puncturing means in the form of puncture section 4 on its distal end. Proximal to the puncture section 4 the puncture wire has a relative flexible section 2. More proximal to this flexible section is a stiff section 3 which extends from the flexible section 2 to the proximal end of the puncture wire 1. The length 21 of the flexible section 2 in the extended situation is 7.5 cm. The length 41 of the cutting section 4 is 5 mm.

The diameter of the wire is 0.81 mm and it has a total length of 180 cm. The puncture wire has a core fabricated from Nitinol and is coated with polyurethane and a hydrophilic polymer. This reduces the friction when advancing the puncture wire through the vasculature. The puncture section 4 is manufactured from stainless steel providing optimal stiffness for puncturing the tissue.

According to the invention, the flexible section 2 is sufficiently flexible allowing advancement of the puncture wire 1 through the vasculature without damaging the walls of the vasculature. When the puncture section 4 would engage the walls of the vasculature during advancement, the flexible section 2 prevents any substantial pressure to be exerted by the puncture section 4 on the wall, preventing any unintended damage. The stiff section 3 facilitates the advancement and steering of the puncture wire 1 and allows advancement of other parts over said wire 1.

In FIG. 1B, the puncture section 4 is shown in more detail. The puncture section 4 is stiff and has a drill-like distal end. The distal end of the puncture section 4 comprises an oblique face 4a under an angle a of 45° with respect to the axis 2a of the puncture wire. The oblique face 4a forms a sharp point 42 allowing puncturing of the septum 105. The puncturing section 4 is also provided with pressure sensing means in the form of an electronic pressure sensor 43. The pressure sensor 43 is connected through the core of the wire to a suitable pressure monitor (not shown).

In another embodiment, the puncture wire according to the invention is hollow. The puncturing section 4 or the distal part of the flexible section 2 is hereby provided with an opening, for instance on the location of sensor 43, allowing fluid communication between the distal end of the puncture wire and a conventional pressure monitor based on hydrostatic pressure connected on the proximal side of the wire.

FIG. 2A schematically shows a heart 1 in cross-section with a right atrium 101 and a left atrium 102. Between the left atrium 102 and right atrium 101 extends a septum 105, comprising the relative flexible fossa ovalis. Also shown is the puncture wire 1 according to the invention for puncturing the septum 105 allowing access to the left atrium 102 through the right atrium 101. In FIG. 1A, the device according to the invention is in the unsupported state, wherein the puncture section 4 is unsupported and is held by the flexible section 2.

The device as shown in FIG. 1A is shown in more detail in FIG. 3. The device furthermore comprises supporting means in the form of a dilator 5 and a sheath 6, both movable over the puncture wire 1. The dilator 5 comprises a tapered end 51 which distal diameter 52 substantially corresponds to the diameter of the puncture wire 1 and the puncturing section 4. The flexible section 2 extends distally from both the sheath 6 and the dilator 5 leaving the flexible length 2 holding the puncturing section 4 at least partially unsupported. Since the puncturing section 4 is unsupported, it is prevented from causing any damage to the vasculature when being advanced. It will be appreciated that the puncture wire 1 according to the invention can be used as a guiding wire in the unsupported state.

It should be noted that although the flexible section 2 is drawn in a curved orientation in FIG. 3, it is also possible to provide the flexible section 2 with sufficient stiffness allowing a more straight orientation, for instance such as shown in FIG. 1A.

Referring back to FIG. 1, the puncture wire 1 is for instance inserted into the vasculature in the groin and advanced in the inferior vena cava 103 and through the right atrium 101 to the superior vena cave 104 in a direction indicated with I. Using the prior art devices, the guide wire should be retracted from the vasculature in this phase of the procedure allowing the advancement of a stiff needle. With the device according to the invention, the puncture wire 1 is retracted proximally inside the supporting means, specifically the dilator 5 as is shown in FIG. 2B and in more detail in FIG. 4. The dilator 5 is moved distally with respect to the sheath 6 such that the dilator 5 extends distally from the sheath 6.

It should be noted that when the terms distal and proximal are used, the directions indicated with D for distal and P for proximal are meant as shown for instance in FIG. 3. And when for instance the dilator 5 is moved distally with respect to the sheath 6 as mentioned earlier, this can be achieved by moving the dilator 5 distally and keeping the sheath 6 in place, but also by proximally retracting the sheath 6 keeping the dilator 5 in place. A combination of the two is also possible.

The device in the configuration as shown in FIGS. 2B and 4 forms a relative stiff combination in comparison to the unsupported state due to the dilator 5 and the sheath 6. This allows the practitioner to move the distal part 52 of the dilator 5 of the combination along the wall 106 of the right atrium 101 towards the septum 105 in a direction II. When the combination is moved over the wall part 106a, the combinations ‘falls’ onto the septum 105 which the practitioner can detect using conventional imaging means, ensuring in a proper placement of the distal end 52 of the dilator 5 on the septum 5.

For easy steering of the dilator/wire combination to the fossa ovalis, the dilator 5 can be provided with a bend. When moving the bent dilator over the wall 106 and over the wall part 106a, the dilator is directed under influence of said bend to the septum. It is also possible to use a steerable sheath for steering the combination as depicted in FIG. 4 onto the septum 105. Using a steerable sheath, the distal part of the sheath, approximately 5 to 10 cm, can be deflected my manipulating a handle provided on the proximal end of the sheath. Said deflection facilitates the steering of the combination onto the septum.

It is an advantage of the device according to the invention that in the case the practitioner missed the ‘jump’ to the septum 105, he can retract the dilator 5 in the sheath 6 exposing the puncture wire 1, preferably while simultaneously moving the sheath 6 caudally. Since the puncture section 4 is then in its unsupported state, the device can be advanced to the superior vena cava 104 again for another try. It will be appreciated that exchanging the needle for a guiding wire according to the prior art devices is not required.

When the combination as shown in FIG. 4 is placed properly on the septum 105, the puncturing section 4 can be moved to the supported state by moving the puncture wire 1 distally with respect to the dilator 5 as is shown in FIG. 5. In this state, the puncturing section 4 extends 3 mm from the distal end 52 of the dilator 5. The puncturing section 4 engages the septum 105 and since the puncturing section 4 is confined by the distal end 52 of the dilator 5, the puncture section 4 is allowed to puncture. The puncturing section 4 is hereby supported by the dilator 5, allowing the puncturing section 4 to exert pressure on the septum 105. The puncturing action is achieved by rotating the puncture wire 1 around its axis as is indicated by the arrow R. This drilling action allows a controlled cutting action through the septum 105, reducing the risk of puncturing the wall of left atrium 102 after puncturing the septum 105. The puncturing action results in a hole or puncture 105a in the septum 105 with a diameter substantially corresponding to the diameter of the puncturing section 4.

After puncturing the septum 105, the puncture wire 1 is advanced distally into the left atrium 102. Since the puncturing section 4 is now in its unsupported state since it is no longer supported by the dilator 5, no damage can be done to the interior of the left atrium 102. The flexible section 2 ensures that the sharp distal end 42 of the puncture section 4 will be harmless in the inside the left atrium 102 or perhaps the left ventricle in case the puncture wire 1 is advanced further. The puncture wire 1 is preferably advanced into the left upper pulmonary vein 107 marking correct puncturing. Since the vein 107 lies outside the contours of the heart when being imaged, it can easily be detected using convention imaging means whether the puncture is successful by locating for instance the puncturing section 4. It is also possible to conduct a pressure measurement for ensuring proper puncturing. It is furthermore possible to introduce contrast fluid through the hollow wire and an opening provided in the distal part of the wire 1 for ensuring proper placement.

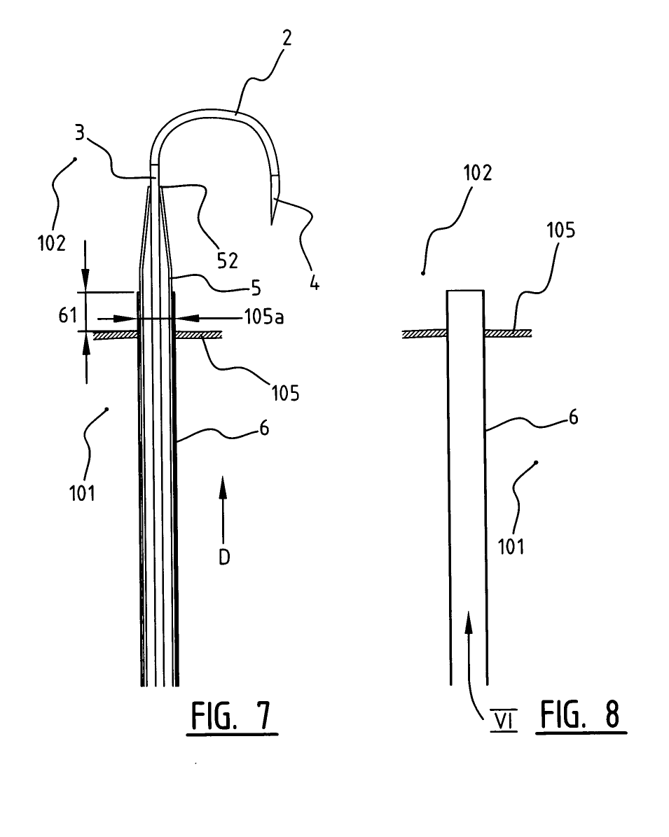

Next, a length 31 of approximately 2 cm of the stiff section 3 is advanced into the left atrium 102 by moving the puncture wire 1 distally. The stiff section 3 supports the dilator 5 in its advancement into the left atrium 102. In this advancement over the stiff section 3, the distal end 52 is forced through the hole 105a in a direction indicated with D and the tapering section 51 of the dilator 5 widens the hole 105a as can be seen in FIG. 7. Also the sheath 6 is advanced into the left atrium 102 for a length 61 of 1 cm.

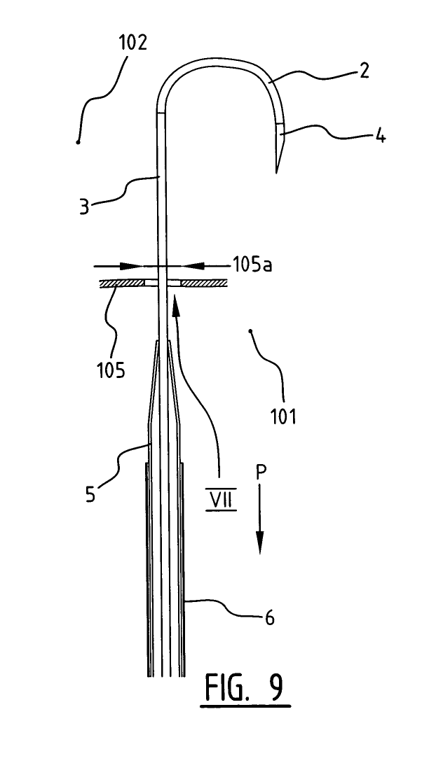

At this point, the dilator 5 and the puncture wire 1 can be retracted from the sheath 6 resulting in the situation as shown in FIG. 8. The sheath 6 which extends through the septum 105 allows a practitioner to advance the needed equipment, for instance a catheter, from the proximal part of the sheath 6 extending outside the patients body through said sheath 6 into the left atrium 102 of the patient, indicated with an arrow VI.

When for instance more than one catheter is needed inside the left atrium 102, instead of retracting the wire 1 and the dilator 5 as is mentioned above, the dilator 5 and the sheath 6 can be moved proximally P back into the right atrium 101 as is indicated in FIG. 9. Since the dilator 5 has widened the hole 105a, it is possible to advance for instance a catheter along the puncture wire 1 indicated with arrow VII into the left atrium 102. The puncture wire 1 then functions as a guide for the catheter, allowing the latter to ‘find’ the puncture 105a and advance from the right atrium 101 into the left atrium 102. In a next step, the dilator 5/sheath 6 combination can be moved distally over the puncture wire 1 resulting in a situation as shown in FIG. 7 but with an extra catheter present in the atrium 102. By retracting the dilator 5 and the wire 1, a second catheter can be introduced into said atrium 102, according to the procedure mentioned earlier.

The invention is not restricted to the variants shown in the drawing, but it also extends to other embodiments that fall within the scope of the appended claims.

1-25. (canceled)

26. A device for use in a medical procedure within the vasculature of a patient, comprising:
puncturing means provided at a distal end of the device for puncturing tissue in a supported state, and;
an elongate member having a proximal end, a distal end provided with the puncturing means and a flexible length therebetween, wherein the elongate member is arranged to be advanced through the vasculature in an unsupported state of the puncturing means without damaging the walls of the vasculature.

27. The device according to claim 26, wherein the elongate member is flexible for a length between 1 to 15 cm, preferably between 2 to 10 cm, and more preferably of approximately 5 cm proximal to the puncturing means.

28. The device according to claim 26, wherein the elongate member proximal to the flexible length comprises a stiff length.

29. The device according to claim 26, wherein the distal length of the elongate member is bent.

30. The device according to claim 26, wherein the puncturing means comprise a stiff puncturing section distal to the flexible length comprising a sharp distal end.

31. The device according to claim 30, wherein the puncturing section has a length between 1 and 10 mm, preferably between 3 and 7 mm, and more preferably of approximately 5 mm.

32. The device according to claim 30, wherein the sharp distal end comprises at least one oblique face under an angle with the axis of the elongate member between 20 and 70 degrees, preferably between 35 and 55 degrees, and more preferably of approximately 45 degrees.

33. The device according to claim 26, wherein the elongate member is provided with pressure sensing means at a distal location.

34. The device according to claim 26, further comprising supporting means, wherein the supporting means and the elongate member are arranged to move between the unsupported state wherein the puncturing means are unsupported and the supported state wherein the puncturing means are supported by the supporting means allowing puncturing of the tissue.

35. The device according to claim 34, wherein the supporting means comprise a tubular member movable over the elongate member, and wherein at least a distal section of the tubular member has an inner diameter suitable for supporting the puncturing means in the supported state.

36. The device according to claim 35, wherein the tubular member is provided with a bend, and wherein the tubular member is bent at an angle with respect to the axis of the elongate member between 25° and 65°, preferably approximately 45°.

37. The device according to claim 34, wherein the supporting means comprise a dilator.

38. The device according to claim 26, further comprising a sheath movable over the elongate member.

39. A method for puncturing tissue in the vasculature of a patient, specifically for puncturing the septum extending between the left and the right atrium of the heart, using a device according to claim 26, comprising the steps of:
advancing the puncturing means provided on the distal end of the elongate member through the vasculature in the proximity of the tissue;
supporting the puncturing means allowing puncturing of the tissue; and
activating the puncturing means for puncturing the tissue.

40. The method according to claim 39, wherein the step of supporting the puncturing means comprises moving a tubular member over and relative to the elongate member to the puncturing means such that the puncturing means extend distally from the tubular member allowing puncturing of the tissue.

41. The method according to claim 40, wherein the puncturing means extend from the tubular member with a length between 0.1 to 10 mm and preferably 1 to 5 mm, and more preferably approximately 3 mm.

42. The method according to claim 39, wherein the step of activating the puncturing means comprises rotating the elongate member.

43. The method according to claim 39, wherein the step of advancing the puncturing means comprises advancing the elongate member through the vasculature whereby the flexible length holding the puncturing means is at least partially unsupported.

44. The method according to claim 40, wherein the step of advancing the puncturing means further comprises the step of advancing the elongate member at least partially in the superior vena cava, retracting the puncturing means in the tubular member and subsequently descending to the septum for puncturing.

45. The method according to claim 39, further comprising the step of at least partially advancing the flexible length of the elongate member through the puncture into the left atrium after puncturing the septum.

46. The method according to claim 45, wherein the elongate member is advanced into a pulmonary vein, preferably the left upper pulmonary vein.

47. The method according to claim 45, wherein the elongate member is advanced in the left atrium until at least a part of the stiff length extends in the left atrium.

48. The method according to claim 40, further comprising the step of at least partially advancing the tubular member through the puncture after puncturing, wherein the tubular member comprises a dilator.

49. The method according to claim 48, further comprising the step of subsequently retracting the dilator from the puncture.

50. The method according to claim 39, further comprising the step of advancing a sheath through the puncture into the left atrium.
